(19) United States
(12) Patent Application Publication
(10) Pub. No.: US 2002/0183489 A1
(43) Pub. Date: Dec. 5, 2002

(54) Large scale production of low fat and SDS gel pure kappa-casein glycomacropeptides (GMP) from bovine deproteinzed whey

(76) Inventors: Martin E. Davis, Tonka Bay, MN (US); Fang Ming, Madison Lake, MN (US); Sharyn X. Su, Plymouth, MN (US); Mengyan Yang, Le Sueur, MN (US); Akimoto Ichinomiya, Tokushima (JP)

Correspondence Address: WARE FRESSOLA VAN DER SLUYS &, ADOLPHSON, LLP, BRADFORD GREEN BUILDING 5, 755 MAIN STREET, P O BOX 224, MONROE, CT, 06468, US

(21) Appl. No.: 10/099,612

(22) Filed: Mar. 14, 2002

Related U.S. Application Data
(60) Provisional application No. 60/275,878, filed on Mar. 14, 2001.

(51) Int. Cl. C07K 1/00

(52) U.S. Cl. 530/361

(57) ABSTRACT

The production of GMP in suitable quantities and of suitable quality for supply to the food, pharmaceutical, cosmetic, and other industries, is provided. The overall cheese making is made more efficient by recovering valuable kappa-casein glycomacropeptides from whey in a manner that permits most whey protein to be separated from the whey prior to concentrating and recovering glycomacropeptides from bovine whey. The invention provides procedures working on concentrated micro-filtered deproteinized whey protein (MFDPW) and obtaining a purified residue which can be dried.

### BACKGROUND OF THE INVENTION

The invention relates to the large scale production of low fat and SDS Gel Pure kappa-casein glycomacropeptides (GMP) from bovine deproteinized whey to enable the production of GMP in suitable quantities and of suitable quality for supply to the food, pharmaceutical, cosmetic, and other industries.

Recently, several biological functions of kappa-casein GMP have been reported, which has encouraged the application of kappa-casein GMP as an ingredient for dietetic foods, health foods and pharmaceuticals. One report in Bulletin of Experimental Biology and Medicine, 98, 889, (1983) showed that dogs were injected with GMP through a fistula exhibited a change in GI motility which may affect food intake. This result suggests that the kappa-casein GMP might be applied as a food material for help in controlling obesity. It was also found that the Kappa-casein GMP could prevent the adhesion of E. coli to intestines' cell and could protect teeth from tartar buildup (Japanese Unexamined Patent Application No. 83-284133/1988. Yamada and Ikeda, et al., (1991) showed a result that kappa-casein GMP could stimulate the proliferation of the human cells. Dosako and Kusano, et al., also claimed that GMP is an active ingredient to prevent the adhesion of E. coli on cells and for the inhibition of transformation of lymphocytes by EBY and also to have strong HI activity against virus.

Kappa-casein can be derived from milk of different species, including bovine milk. Bovine kappa-casein GMP is peptide-bonded sialic acid, and it is a hydrolysate of bovine milk kappa-casein due to the reaction of chymosin (rennet or pepsin) on κ-casein. Eigel, et al., (1984) considered that the molecular weight of kappa-casein GMP should be 7000 Daltons because the kappa-casein, which has a molecular weight of 19,000 Daltons is cleaved at the Phe-105-Met-106 bond. McKenzie (1971) confirmed that at pH 8.6, 7M urea buffer, the kappa-casein GMP has a molecular weight of 7000 Daltons. Morr and Seo (1988) found that the molecular weight of kappa-casein is 33000 Daltons at pH 7.5, 0. 1M Tris-HCl buffer. In this case, the GMP molecular weight is even larger than that of kappa-casein. Morr and Seo explained that the discrepancy may be due to the bulky nature of carbohydrate moiety of the GMP or to the peptide-peptide interaction that was common to intact kappa-casein in nondissociating buffers. Tanimoto, et al., (1990) reported that at pH 4 the molecular weight of kappa-casein glycomacropeptides is sharply changed. At the pH 4 or lower, the GMP is in the form of a monomer, and has a molecular weight of 9000 Daltons. However, at the pH 5 or higher, the GMP is in the form of a polymer, and has a molecular weight of 50,000 Daltons.

Because the GMP is a hydrolysate of kappa-casein, the GMP concentration in the whey is mainly dependent on the cheese processing from which the whey is obtained, i.e. type of cheese, processing conditions, etc. Typically, liquid whey has total solids content around 6%, and contains about 94% water. Lactose is typically present at a concentration of about 4.3%, lactic acid about 0.2%, ash about 0.5% and fat about 0.15%. Whey protein (total nitrogen times 6.38) is around 0.85%. In the whey most of the total nitrogen is due to protein, but a small fraction is non-protein nitrogen. The non-protein nitrogen of whey comprises GMP and other nitrogenous compounds. The whey protein comprises beta-lactoglobulin, alpha-lactalbumin, bovine serum albumin, immunogluobulins, lactoferrin and lactoperoxidase. In addition, aggregated protein, which is generated during cheese and whey processing, is also in the whey. The separation of the GMP component, while maintaining recovery of other useful components, presents a technical challenge. The challenge to do this on a large, commercial scale is even greater.

A number of separation techniques might be applied in a large scale to produce GMP. Membrane separation techniques are among them. However, membrane processing has a lower selectivity in comparison with ion exchange processing. This could lead to a low purity of product. Therefore, membrane processing is usually employed for concentration and diafiltration of the isolated GMP. Both cation and anion exchangers can be applied for GMP separation. An anion-exchanger, DEAE resin, is usually applied to adsorb GMP at pH 7.5, and then the GMP is desorbed by increasing the salt concentration in eluting solution. GMP separation is also possible by applying a cation-exchanger to remove it from the other proteins. However, fat and aggregated protein cannot be adsorbed by the cation exchanger in this manner, and this type of processing could lead to a high fat and low purity form of GMP.

Tanimoto and Kawasaki, et al., (1990) reported that they were able to achieve a yield of 6.5 g GMP with a purity of 78% from 500 liters of Gouda cheese whey by adjusting the whey pH and then concentrating by UF membrane. By using a similar processing method, Tanimoto and Kawasaki, et al., (1990) prepared a whey protein solution by using 1 kg whey protein concentrate and dissolving it in 50 liters of water. They reported a yield 54g GMP with purity of 80%.

Morimasa and Yoshihiro, et al., (1992) used 10 kg of lactic casein to prepare rennet casein whey. From 100 liters of the above rennet casein whey, they obtained 180 g of crude GMP by using membrane and reverse osmosis (RO) processing.

Kawagoe and Urawa (1994) applied cation exchanger with carboxy methyl group, diethyl amino group or sulfone group to separate GMP form whey. They claimed that the obtained GMP was 82% pure based on a urea-SDS electrophoresis method.

There is a current need for a process which is capable of large-scale production of low fat and SDS gel pure kappa-casein glycomacropeptides (GMP) from bovine deproteinized whey.

### BRIEF DESCRIPTION OF THE INVENTION

It is an objective of the invention to provide methodology suitable for obtaining GMP from deproteinized whey.

It is another object of the invention to enable the production of kappa-casein glycomacropeptides in suitable quantities and of suitable quality for supply to the food, pharmaceutical, cosmetic, and other industries.

It is another object of the invention to produce SDS gel pure and HPLC pure GMP from deproteinized whey protein (DPW).

It is another object of the invention to provide procedures for working on concentrated micro-filtered deproteinized whey protein (DPW) and obtaining a purified residue which can be dried.

It is another object of the invention to improve the overall cheese making process by recovering valuable glycomacropeptides from whey in a manner that permits most whey protein to be separated from the whey prior to concentrating and recovering the GMP present in the whey.

These and other objects are accomplished by the present invention by providing a process for producing an SDS gel pure and low fat kappa-GMP, in which both membrane and ion exchange chromatogram techniques are applied.

In one aspect, the process provides a process for preparing GMP from bovine whey, comprising: processing bovine whey to remove fat, whey protein and aggregated proteins to produce a deproteinized whey (DPW); concentrating the DPW; acidifying the DPW; at acid pH, contacting the DPW with an ion exchange resin to remove non-GMP peptides and proteins to obtain a resin effluent; subjecting the resin effluent to diafiltration to remove lactose, small peptides and minerals to provide a purified resin effluent; concentrating and drying the resin effluent.

Some preferred aspects of the invention are set forth below.

### BRIEF DESCRIPTION OF THE DRAWINGS

The invention will be more fully described and its advantages will become more apparent in view of the following description, especially when read in conjunction with the accompanying drawings wherein:

FIG. 1 is a photograph of an SDS gel (16.5% SDS-Tris-tricine PAGE) having the following three tracks: (1) Low molecular weight markers (26K, 17K, 14.4K, 6.5K); (2) Sigma GMP; and the product GMP in example 2, below.

FIG. 2 is a graph illustrating the HPLC profile for the product GMP in example 2, below.

FIG. 3 is a graph illustrating the HPLC profile for a commercial GMP.

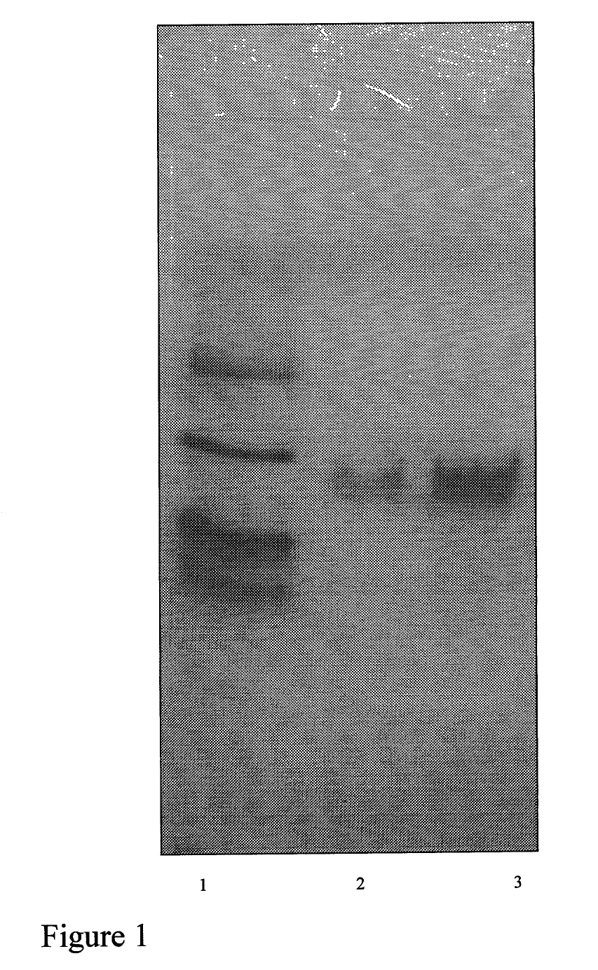

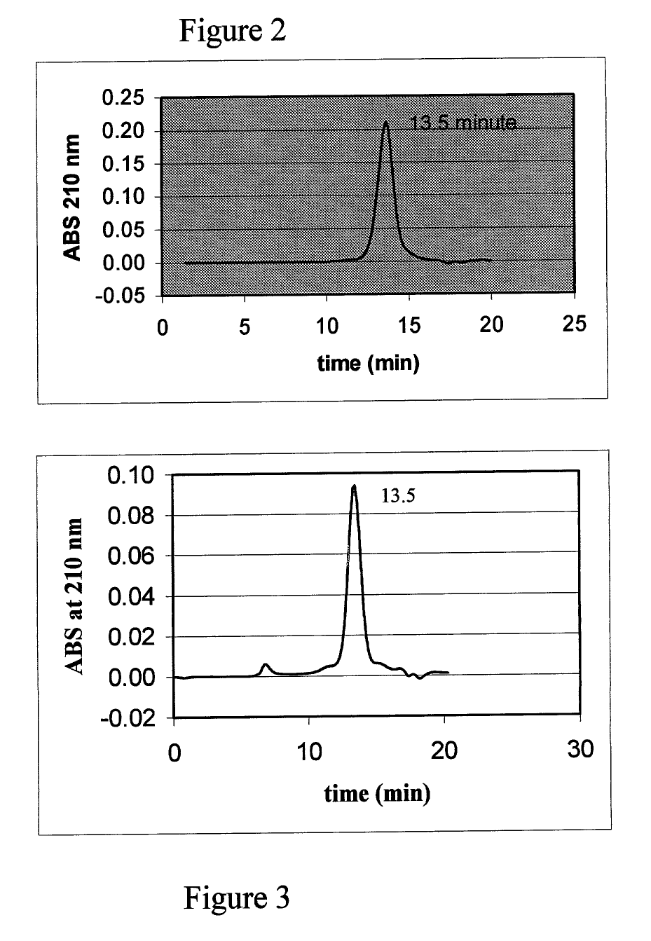

### DETAILED DESCRIPTION

In the following description, DPW means “deproteinized whey”, which is the liquid remaining after treatment of whey to remove the majority of the whey proteins. The material is not deproteinized completely, but contains GMP and other residual proteins from MFGM (milk fat globular membrane) originally present in the whey.

It is an advantage of the invention that the process can produce SDS gel pure and HPLC pure GMP from DPW.

It is another advantage of the invention that it is now possible to produce a very high purity of GMP as compared to a process employing only cation exchange to separate GMP from whey or DPW. The purity obtainable by the invention can be characterized as greater than 91% as measured by HPLC, and preferably greater than 95%.

Advantageously, the process of the invention should include the following three techniques in combination (1) macrofilteration membrane process is applied to remove fat and aggregated protein at pH 3.6; (2) cation exchange is applied to remove residual whey protein at pH 3.3 and (3) ultrafiltration membrane processing is applied to remove lactose, small peptides and minerals at pH 7.0. In a preferred form of the invention, the macrofilteration membrane should be operated at pH 3.6-4.0 to filter GMP out and maintain the fat and aggregated protein in the membrane retentate.

Fresh deproteinized whey (DPW) was obtained from a commercial production of cheddar cheese utilizing bovine milk. The DPW was prepared from whey protein that was contacted with ion-exchanger to adsorb whey protein in the production of a commercial whey protein concentrate. A preferred form of DPW is that obtained from the preparation of full-fat, reduced-fat or fat-free cheese from bovine milk using a chymosin, for example, rennet. A preferred form of ion exchange resin suitable for use in present invention is a cellulose ion exchanger having sulfonic acid groups.

Processing of bovine whey according to the invention entails treating, such as by centrifuge, ion-exchanger or microfiltration to remove fat, whey protein and aggregated proteins (e.g., having a size above about 400,000 Daltons) to produce a deproteinized whey (DPW) suitable for further processing. The pH in DPW is reduced, such as by adjusting to a pH of about 3.5 to 4, and then the DPW is passed through a microfiltration (MF) membrane to remove fat, aggregated protein, and to concentrate GMP. Significantly, the microfiltration membrane should be of a material and porosity to achieve the essentially complete separation of fat, large molecular material, as well as small molecular weight materials. Exemplary of suitable membranes are those produced by Snyder Filtration, available as 0.1 micro MF membrane.

A permeate is recovered from the microfiltration procedure. The MF permeate can be referred to as MFDPW, which means “deproteinized whey” that has been further processed by microfiltration. The MFDPW can be concentrated as above or otherwise to achieve a desired concentration of from about 0.1 to about 6 weight % solids. The MFDPW is applied to a suitable ion exchange, e.g., cation exhange, resin to remove the non-GMP peptides and protein at acid pH, e.g., pH 3.2-3.4. Exemplary of suitable ion exchange resins, e.g., SP resins, are those produced by Life Technology, NZ and available as SP resins, including SP-sepharose resins.

The effluent of the SP resin treatment is collected and the pH is adjusted back to neutral, i.e., about 7. A diafiltration processing by using UF membrane is applied to remove lactose, small peptides and minerals from the SP resin effluent. The diafiltration apparatus is characterized by 3K, 5K or 10K UF membrane. Exemplary of suitable diafiltration apparatus are those produced by Snyder Filtration, CA and available as Ultrafiltration membranes.

The final UF concentrate is spray dried. A low fat, high sialic acid content GMP powder is obtained. The purity of GMP is SDS gel and HPLC pure. Alternatively, the MF membrane processing could be applied after the ion exchange processing. Table 1 shows the average parameters for the GMP produced by the above mentioned processing.

TABLE 1

| Chemical/Physical Parameters | Value |
|---|---|
| Fat | Not more than 0.2% |
| Ash | 6.0-7.0% |
| Lactose | Not more than 1.0% |
| GMP in powder (dry base) | More than 92.0% |
| Purity of GMP | SDS gel and HPLC pure |
| Sialic acid in GMP | More than 10% |

The following examples are presented to further illustrate and explain the invention and should not be taken as limiting in any regard. Unless otherwise indicated, all parts and percentages are by weight.

### EXAMPLE 1

A batch of 100 L of DPW was obtained from a commercial cheese plant, and it was added into SP resin reactor at pH 3.3 to adsorb the residual whey protein. The reactor effluent was collected and pH was adjusted to 7. A 0.1 micron membrane was applied to remove fat and aggregated protein. The permeate from the MF membrane was pumped into a 10K membrane to remove the lactose, small peptides and mineral by diafiltration. After spray drying, 1.8 kg GMP powder was obtained. Table 2 shows the GMP powder composition.

TABLE 2

| Chemical/Physical Parameters | Value |
|---|---|
| Fat | 0.2% |
| Ash | 6.1% |
| Lactose | 0.7% |
| GMP in powder (DB) | 92.6% |
| Purity of GMP | SDS gel and HPLC pure |
| Sialic acid in GMP | 10.9% |

### EXAMPLE 2

A batch of 377 L of UF concentrated DPW with total protein of 3.7% (total N×6.38) was obtained from a commercial cheese plant. The pH was adjusted to 3.6, and then was pumped into a 0.1 micron membrane system to remove fat and aggregated protein. The permeate was collected and then was pumped into an SP resin reactor to remove the residual whey protein at pH 3.3. The effluent from the SP resin reactor was collected, and the pH was adjusted to 7.0. A 10 K membrane was applied to remove the lactose, small peptides and mineral by diafiltration. The 10 K concentrated material was sent to a spray dried. A 2.23 kg product of GMP powder was obtained.

Table 3 provides the details obtained by analysis of the recovered powder.

TABLE 3

| Chemical/Physical Parameters | Value |
|---|---|
| Mositure | 3.80% |
| Fat | 0.08% |
| Ash | 6.20% |
| Lactose | 0.70% |
| GMP in powder (DB) | 93.50% |
| Purity of GMP | SDS gel and HPLC pure |
| Sialic acid in GMP | 11.30% |

The above description is intended to enable the person skilled in the art to practice the invention. It is not intended to detail all of the possible modifications and variations which will become apparent to the skilled worker upon reading the description. It is intended, however, that all such modifications and variations be included within the scope of the invention which is seen in the above description and otherwise defined by the following claims. The claims are meant to cover the indicated elements and steps in any arrangement or sequence which is effective to meet the objectives intended for the invention, unless the context specifically indicates the contrary.

### REFERENCES

1. Eigel, W. N. Butler, J. E., Ernstrom, C. A. Farrll, H. M. Harwallker, V. R. Jennesss, R.,and Whitney, R. McL., 1984, “Nomenclature of proteins of cow's milk: fifth revision “J. Dairy Sci. 67:1599.

2. McKenzie, H. A. 1971, “Composition, Physicochemical And Functional Properties Of Reference Whey Protein Concentrates”. J. Food Sci. 50: 1406.

3. Morr, C.V. and Seo, A., 1988 ” Fractionation And Characterization Of Glycomacropeptide From Caseinate And Skim Milk Hydrolysates”, J. Food Sci., 53: 80.

4. Tanimoto, M., Kawasaki, Y., Sanralzu, H., Dosako, S. and Tomizawa, A., 1990 “Process For Producing Kappa-Casein Glycomacropeptides”. European Patent Application. No. 90303044.8.

5. Yamada, K., Ikeda, I., Nakajima, H., Shirahata, S. and Murakami, H., (1991) “Stimulation Of Proliferation And Immunoglobulin Production Of Human-Human Hybridomas By Various Types Of Caseins And Their Protease Digests”, Cytotechnology 5: 279-285.

6. Shunichi Dosako, Urawa: Hiroko Kusano, Tokorozawa; Eiki Deya, Sayama and Tadashi Idota, Kawagoe, 1992 “Infection Protectant” U.S. Patent. No. 5,147,853.

7. Yoshihiro Kawasaki, Kawaggoe and Shunichi Dosako, Urawa (1994) “Process for Producing kappa-casein glycomacropeptides” U.S. Pat. No. 5,278,288.

1. A process for preparing GMP from bovine whey, comprising:
processing bovine whey to remove fat, whey protein and aggregated proteins to produce a deproteinized whey;
concentrating the DPW;
acidifying the DPW;
at acid pH contacting the DPW with an ion exchange resin to remove non-GMP peptides and proteins to obtain a resin effluent;
neutralizing the resin effluent;
subjecting the resin effluent to microfiltration to remove aggregated protein and fat;
subjecting the resin effluent to diafiltration to remove lactose, small peptides and minerals to provide a purified resin effluent;
concentrating and drying the resin effluent.

2. A process for preparing GMP from bovine whey, comprising:
processing bovine whey to remove fat, whey protein and aggregated proteins to produce a deproteinized whey;
concentrating the DPW;
subjecting the resin effluent to microfiltration to remove aggregated protein and fat;
acidifying the microfiltration permeate;
at acid pH, contacting the microfiltration permeate with an ion exchange resin to remove non-GMP peptides and proteins to obtain a resin effluent;
neutralizing the resin effluent;
subjecting the resin effluent to diafiltration to remove lactose, small peptides and minerals to provide a purified resin effluent; and
concentrating and drying the resin effluent.

3. GMP from bovine deproteinized whey having an SDS Gel Purity of at least 91%.

4. GMP according to claim 3 having an SDS Gel Purity of at least 95%.

5. GMP from bovine deproteinized whey produced by the process of either claim 1 or 2.

6. GMP according to claim 5 having an SDS Gel Purity of at least 91%.
